(19) United States
(12) Patent Application Publication
(10) Pub. No.: US 2010/0062058 A1
(43) Pub. Date: Mar. 11, 2010

(54) Delayed Release Formulations for Oral Administration of a Polypeptide Therapeutic Agent and Methods of Using Same

(75) Inventors: Nicholas W. Warne, Andover, MA (US); Rebecca Koval, Billerica, MA (US); Arwinder S. Nagi, Thiells, NY (US); Ramarao S. Chatlapalli, Hopewell Junction, NY (US); Eric J. Benjamin, Jamestown, NC (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Wyeth, Madison, NJ (US)

(21) Appl. No.: 12/420,727

(22) Filed: Apr. 8, 2009

Related U.S. Application Data
(63) Continuation of application No. 10/663,264, filed on Sep. 16, 2003, now abandoned.
(60) Provisional application No. 60/411,040, filed on Sep. 16, 2002.

(51) Int. Cl. A61K 38/20; A61K 9/24; A61K 9/52; A61P 7/00; A61P 29/00

(52) U.S. Cl. 424/456; 424/85.2; 424/472; 424/457

(57) ABSTRACT

The invention provides compositions containing polypeptides, including therapeutic polypeptides such as interleukin-11, that are suitable for oral administration.

### RELATED APPLICATIONS

This application claims priority to U.S. Ser. No. 60/411,040, filed Sep. 16, 2002. The contents of this application are incorporated herein by reference in their entirety.

### FIELD OF THE INVENTION

The invention relates to compositions containing polypeptides, including interleukin-11, that are suitable for oral administration.

### BACKGROUND OF THE INVENTION

Recombinant human interleukin-11 (rhIL-11) is a non-glycosylated polypeptide of 177 amino acids. The polypeptide lacks cysteine residues and is highly basic (pI>10.5). rhIL-11 is a member of a family of human growth factors that includes human growth hormone (hGH) and granulocyte colony-stimulating factor (G-CSF).

rhIL-11 is used as a chemotherapeutic support agent and is administered in conjunction with other cancer treatments to increase platelet levels. rhIL-11 has also been demonstrated to have anti-inflammatory effects and to be useful in treating conditions such as Crohn's disease and ulcerative colitis. IL-11 is typically administered via subcutaneous injection. Formulations for subcutaneous injections must be sterile, and can be expensive relative to other routes of administration. The route is also inconvenient and uncomfortable. Subcutaneous injection has additionally been associated with complications such as local tissue damage and infection at the area of injection.

### SUMMARY OF THE INVENTION

The invention is based in part on the discovery of rhIL-11 compositions that can be delivered orally to a subject.

In one aspect, the invention provides a therapeutically effective delayed release oral dosage composition that includes a bioactive polypeptide, an enteric coat (such as a methacrylic acid copolymer), and, optionally, at least one excipient. In some embodiments, the bioactive polypeptide includes one or more properties selected from the group consisting of lacking an N-linked glycosylation site, having no more than one cysteine amino acid, and having a basic pI. In some embodiments, the polypeptide has no cysteine residues.

A preferred polypeptide is IL-11. The invention is described herein with reference to the bioactive polypeptide IL-11. However, it is understood that the features of the invention described with respect to IL-11 are also applicable to compositions and methods including other bioactive polypeptides

In one embodiment, the composition further includes an inert core. The inert core can be, e.g., a pellet, sphere or bead made up of sugar, starch, microcrystallinecellulose or any other pharmaceutically acceptable inert excipient. A preferred inert core is a carbohydrate, such as a monosaccharide, disaccharide, or polysaccharide, i.e., a polymer including three or more sugar molecules. An example of a suitable carbohydrate is sucrose. In some embodiments, the sucrose is present in the composition at a concentration of 60-75% wt/wt.

When the bioactive polypeptide is IL-11, the IL-11 layer is preferentially provided with a stabilizer such as methionine, glycine, polysorbate 80 and phosphate buffer, and/or a pharmaceutically acceptable binder, such as hydroxypropyl methylcellulose, povidone or hydroxypropylcellulose. The composition can additionally include one or more pharmaceutical excipients. Such pharmaceutical excipients include, e.g., binders, disintegrants, fillers, plasticizers, lubricants, glidants, coatings and suspending/dispersing agents.

A preferred binder is hydroxypropyl methylcellulose (HPMC). The HPMC is preferably present in the composition at a concentration of 3-7% wt/wt.

A preferred glidant is talc. In some embodiments, the glidant is present in the composition at a concentration of 5-10% wt/wt.

Plasticizers can include, e.g., triethylcitrate, polyethylene glycols, dibutyl phthalate, triacetin, dibutyl sebucate and propylene glycol. A preferred plasticizer is triethyl citrate. For example, the triethyl citrate can be present at a concentration of 1-2% wt/wt.

A preferred surfactant is polysorbate 80. The polysorbate 80 can be present at a concentration of 0.015-0.045% wt/wt.

In some embodiments, the composition is provided as a multiparticulate system that includes a plurality of enteric coated, IL-11 layered pellets in a capsule dosage form. The enteric coated IL-11 pellets include an inert core, such as a carbohydrate sphere, a layer of IL-11 and an enteric coat. The enteric coat can include, e.g., a pH dependent polymer, a plasticizer, and an antisticking agent/glidant. Preferred polymers include, e.g., methacrylic acid copolymer, cellulose acetate phthalate, hydroxypropylmethylcellulose phthalate, polyvinyl acetate phthalate, shellac, hydroxypropylmethylcelluloseacetate succinate, carboxy-methylcellulose.

Preferably, an inert seal coat is present in the composition as a barrier between the IL-11 layer and enteric coat. The inert seal coat can be, e.g., hydroxypropylmethyl cellulose, povidone, hydroxypropylcellulose or another pharmaceutically acceptable binder.

Suitable sustained release polymers include, e.g., amino methacrylate copolymers (Eudragit RL, Eudragit RS), ethylcellulose or hydroxypropyl methylcellulose. In some embodiments, the methacrylic acid copolymer is a pH dependent anionic polymer solubilizing above pH 5.5. The methacrylic acid copolymer can be provided as a dispersion and be present in the composition at a concentration of 10-20% wt/wt. A preferred methacrylic acid copolymer is EUDRAGIT® L 30 D-55.

In preferred embodiments, the enteric coated tablet dosage form includes IL-11, a filler microcrystallinecellulose (Avicel PH 102), a disintegrant Explotab, a buffer sodium phosphate, an antioxidant methionine, a surfactant Tween 80, a lubricant magnesium stearate and an enteric coat.

In a preferred embodiment, the sustained release tablet dosage form that includes IL-11, fillers (e.g., microcrystallinecellulose (Avicel PH 102) and sucrose), a matrix forming polymer (hydroxypropylmethylcellulose Methocel K4M Prem, Methocel K100 LV, LH, CR, Premium), a glidant (such as Syloid), a buffer sodium phosphate, an antioxidant methionine, a surfactant (such as Tween 80), and a lubricant (such as magnesium stearate).

In another embodiment, the composition includes glycine. In some embodiments, the glycine is present in the composition at a concentration of 1-4% wt/wt.

The composition may optionally further include an antioxidant. An example of a suitable antioxidant is methionine. In some embodiments, the methionine is present in the composition at a concentration of 0.1-0.5% wt/wt.

The IL-11 can be provided as a purified protein isolated from naturally occurring IL-11. Alternatively, the IL-11 polypeptide can be provided as a recombinant form of the polypeptide, e.g., recombinant human IL-11 (rhIL-11).

In another aspect, the invention provides a therapeutically effective delayed release oral dosage multiparticulate composition including an IL-11 polypeptide, a first sealing coat, an enteric coating layer, and a second sealing coat. A preferred sealing coat is HPMC. The enteric coating layer of the composition can be, e.g., a methacrylic acid copolymer. A preferred methacrylic acid copolymer is soluble at a pH above 5.5, for example EUDRAGIT® L 3

Also provided by the invention is a sustained release composition that includes an IL-11 polypeptide, an enteric coat (such as a methacrylic acid copolymer), and, optionally, at least one excipient. In one embodiment, the composition further includes an inert core. The inert core can be, e.g., a pellet, sphere or bead made up of sugar, starch, microcrystallinecellulose or any other pharmaceutically acceptable inert excipient. A preferred inert core is a carbohydrate, such as a monosaccharide, disaccharide, or polysaccharide, i.e., a polymer including three or more sugar molecules. An example of a suitable carbohydrate is sucrose. In some embodiments, the sucrose is present in the composition at a concentration of 60-75% wt/wt.

The invention also provides a method of delivering an IL-11 polypeptide to a subject by orally administering to the subject an IL-11 polypeptide containing composition as described herein in an amount sufficient to elicit a biological response in the subject. In some embodiments, the response is elicited in the small intestine of the subject.

The subject used in the herein described method can be, e.g., a human, a non-human primate, a dog, a cat, horse, cow, pig, sheep, rabbit, rat, or mouse.

In another aspect, the invention provides a method of treating or preventing inflammation in a subject by administering to the subject an oral composition that includes IL-11. In some embodiments, the inflammation is associated with ulcerative colitis and Crohn's disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a schematic diagram of a multi-particulate IL-11 formulation suitable for oral delivery.

FIG. 2 is a schematic illustration of a process for making a multi-particulate IL-11 formulation suitable for oral delivery.

### DETAILED DESCRIPTION OF THE INVENTION

The invention provides formulations of bioactive polypeptides that are suitable for oral delivery. In some embodiments, the bioactive polypeptide is non-glycosylated (e.g., lacking either N-linked or O-linked glycosylation sites, or both sites), lacks a cysteine residue, and/or has a basic pI. The absence of glycosylation can be either because the naturally occurring polypeptide lacks sites for glycosylation or because the protein has been engineered to lack these sites. Alternatively, the polypeptide may be treated with, e.g., glycosylases to reduce or remove glycosylated residues. Similarly, the lack of cysteine residues can occur in the naturally occurring polypeptide sequence or in a variant form of a polypeptide in which naturally occurring cysteine residues have been either deleted or replaced with non-cysteine residues.

A preferred polypeptide for use in the formulation is interleukin 11 (IL-11). This protein is a pleiotropic cytokine that stimulates primitive lymphohematopoietic progenitor cells and acts in synergy with other hematopoietic growth factors to stimulate the proliferation and maturation of megakaryocytes. IL-11 is described in detail in International Application PCT/US90/06803, published May 30, 1991; as well as in U.S. Pat. No. 5,215,895; issued Jun. 1, 1993. A cloned human IL-11 was previously deposited with the ATCC, 10801 University Boulevard, Manassas, Va. 20110-2209, on Mar. 30, 1990 under ATCC No. 68284. Moreover, as described in U.S. Pat. No. 5,270,181; issued Dec. 14, 1993; and U.S. Pat. No. 5,292,646; issued Mar. 8, 1994; IL-11 may also be produced recombinantly as a fusion protein with another protein. IL-11 can be produced in a variety of host cells by resort to now conventional genetic engineering techniques. In addition, IL-11 can be obtained from various cell lines, for example, the human lung fibroblast cell line, MRC-5 (ATCC Accession No. CCL 171) and Paul et al., the human trophoblastic cell line, TPA30-1 (ATCC Accession No. CRL 1583). Described in Proc Natl Acad Sci USA 87:7512 (1990) is a cDNA encoding human IL-11 as well as the deduced amino acid sequence (amino acids 1 to 199). U.S. Pat. No. 5,292,646, supra, describes a des-Pro form of IL-11 in which the N-terminal proline of the mature form of IL-11 (amino acids 22-199) has been removed (amino acids 23-199). As is appreciated by one skilled in the art, any form of IL-11, which retains IL-11 activity, is useful according to the present invention.

In addition to recombinant techniques, IL-11 may also be produced by known conventional chemical synthesis. Methods for constructing the polypeptides useful in the present invention by synthetic means are known to those of skill in the art. The synthetically constructed cytokine polypeptide sequences, by virtue of sharing primary, secondary, or tertiary structural and conformational characteristics with the natural cytokine polypeptides are anticipated to possess biological activities in common therewith. Such synthetically constructed cytokine polypeptide sequences or fragments thereof, which duplicate or partially duplicate the functionality thereof may also be used in the method of this invention. Thus, they may be employed as biologically active or immunological substitutes for the natural, purified cytokines useful in the present invention.

Modifications in the protein, peptide or DNA sequences of these cytokines or active fragments thereof may also produce proteins which may be employed in the methods of this invention. Such modified cytokines can be made by one skilled in the art using known techniques. Modifications of interest in the cytokine sequences, e.g., the IL-11 sequence, may include the replacement, insertion or deletion of one or more selected amino acid residues in the coding sequences. Mutagenic techniques for such replacement, insertion or deletion are well known to one skilled in the art. (See, e.g., U.S. Pat. No. 4,518,584.)

Other specific mutations of the sequences of the cytokine polypeptides which may be useful therapeutically as described herein may involve, e.g., the insertion of one or more glycosylation sites. An asparagine-linked glycosylation recognition site can be inserted into the sequence by the deletion, substitution or addition of amino acids into the peptide sequence or nucleotides into the DNA sequence. Such changes may be made at any site of the molecule that is modified by addition of O-linked carbohydrate. Expression of such altered nucleotide or peptide sequences produces variants which may be glycosylated at those sites.

Additional analogs and derivatives of the sequence of the selected cytokine which would be expected to retain or prolong its activity in whole or in part, and which are expected to be useful in the present method, may also be easily made by one of skill in the art. One such modification may be the attachment of polyethylene glycol (PEG) onto existing lysine residues in the cytokine sequence or the insertion of one or more lysine residues or other amino acid residues that can react with PEG or PEG derivatives into the sequence by conventional techniques to enable the attachment of PEG moieties.

Additional analogs of these selected cytokines may also be characterized by allelic variations in the DNA sequences encoding them, or induced variations in the DNA sequences encoding them. It is anticipated that all analogs disclosed in the above-referenced publications, including those characterized by DNA sequences capable of hybridizing to the disclosed cytokine sequences under stringent hybridization conditions or non-stringent conditions (Sambrook et al., Molecular Cloning. A Laboratory Manual, 2d edit., Cold Spring Harbor Laboratory, New York (1989)) will be similarly useful in this invention.

Also considered useful in the compositions and methods disclosed herein are fusion molecules, prepared by fusing the sequence or a biologically active fragment of the sequence of one cytokine to another cytokine or proteinaceous therapeutic agent, e.g., IL-11 fused to IL-6 (see, e.g., methods for fusion described in PCT/US91/06186 (WO92/04455), published Mar. 19, 1992). Alternatively, combinations of the cytokines may be administered together according to the method.

Thus, where in the description of the methods of this invention IL-11 is mentioned by name, it is understood by those of skill in the art that IL-11 encompasses the protein produced by the sequences presently disclosed in the art, as well as proteins characterized by the modifications described above yet which retain substantially similar activity.

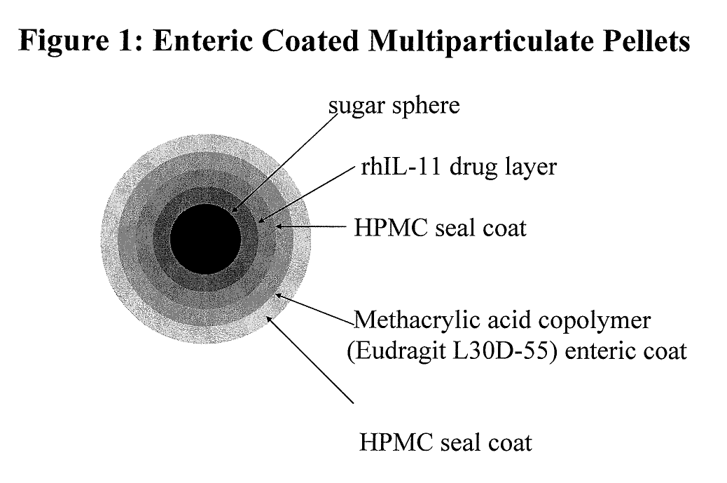

A schematic diagram showing a preferred multiparticulate IL-11 formulation is shown in FIG. 1. On to a central sugar sphere-is disposed a layer containing rhIL-11. This rhIL-11 drug layer in turn is covered with a hydroxypropyl methylcellulose (HPMC) sealing coat. This HPMC sealing coat is covered with a methacrylic acid copolymer (e.g., with Eudragit L20D-55) enteric coat, and the entire pellet is covered with a second or final HPMC sealing coat.

Oral IL-11 formulations can be prepared using any method known in the art. Examples of suitable methods include fluid bed spraying onto sucrose spheres, direct compression, and wet granulation synthetic methods. Methods of preparing compositions according to the invention are illustrated in the Examples, below.

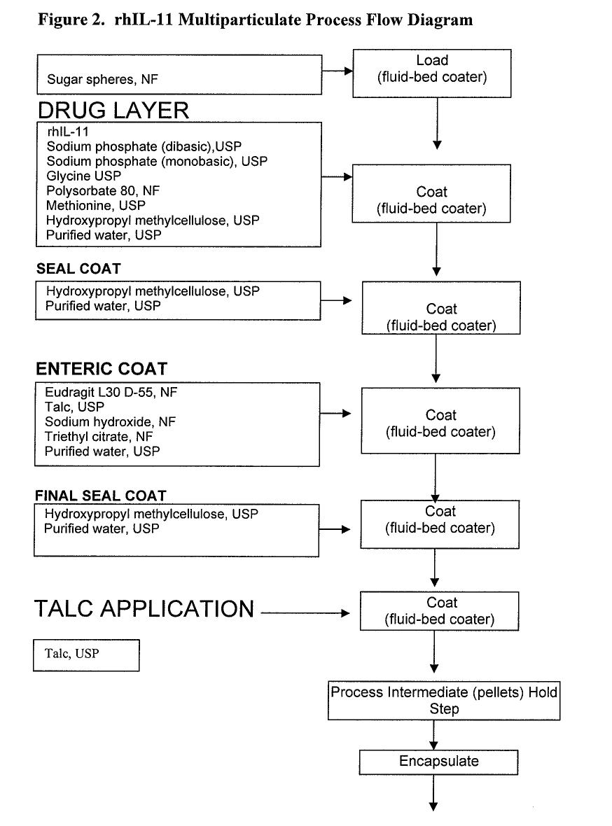

A flow diagram illustrating a preferred method for making multiparticulate IL-11 particles suitable for oral delivery is shown in FIG. 2. The drug layer sealing coat, enteric coat, and second sealing coat are sequentially added within a fluid-bed coater. At each step temperature and mass of the formulations are preferably monitored.

The flow diagram illustrates that sugar spheres are loaded onto a fluid-bed coater and coated with a drug layer that includes rhIL-11, sodium phosphate dibasic, sodium phosphate monobasic, glycine, polysorbate 80, methionine, hydroxypropyl methylcellulose (HPMC), and purified water to form a coat. An enteric coat is applied containing Eudragit, talc, sodium hydroxide, triethyl citrate, and purified water. A seal coat of HPMC and purified water is then applied followed by talc as an anti-static agent. Subsequent processing can include, e.g., storage for 180 days at 2-8 degrees Centigrade.

Procedures for synthesizing formulations suitable for oral delivery are known in the art and are described in, e.g., Bergstrand et al., U.S. Pat. No. 6,428,810, Chen et al., U.S. Pat. No. 6,077,541, Ullah et al., U.S. Pat. No. 6,331,316, Chen et al., U.S. Pat. No. 6,174,548, and Anderson et al., U.S. Pat. No. 6,207,682.

The formulations of the invention can be delivered in any suitable form, e.g., they can be provided as capsules, sachets, tablets or suspensions.

The formulations can be used to treat indications for which IL-11 has been demonstrated to be efficacious. A preferred indication is inflammatory bowel disease (IBD). This condition is characterized by chronic intestinal inflammation that results in clinical symptoms such as diarrhea, bleeding, abdominal pain, fever, joint pain, and weight loss. These symptoms can range from mild to severe, and may gradually and subtly develop from an initial minor discomfort, or may present themselves suddenly with acute intensity.

IBD is a prevalent cause of chronic illness in a large segment of the patient population. It can manifest itself in two different forms: Ulcerative Colitis (UC) and Crohn's Disease (CD). Although the two conditions can appear clinically very similar, UC primarily involves inflammation of the colon and rectum, as opposed to the upper GI tract. Crohn's Disease, in contrast, impacts a greater area of the upper intestinal digestive tract, and is thus more likely to trigger malabsorption, along with chronic vitamin and nutrient deficiencies.

The oral IL-11 formulations described herein can be administered with additional agents that treat inflammatory bowel disease. Additional agents include, e.g., corticosteroids, immunosuppressive agents, infliximab, and mesalamine, which is a substance that helps control inflammation. Mesalamines include, e.g., sulfasalazine and 5-ASA agents, such as Asacol, Dipentum, or Pentasa. The oral IL-11 formulations can additionally be administered with antibiotics, including, for example, ampicillin, sulfonamide, cephalosporin, tetracycline, or metronidazole.

The dosage regimen involved in a method for treating the above-described conditions will be determined by the attending physician considering various factors which modify the action of drugs, e.g. the condition, body weight, sex and diet of the patient, the severity of any infection, time of administration and other clinical factors. Generally, the daily regimen should be in the range of 1-30 milligrams of polypeptide.

The invention will be further illustrated in the following non-limiting examples.

### Example 1

### Compatibility of rhIL-11 with Various Formulation Excipients and Antioxidants

Compatibility studies were performed on rhIL-11 tablets containing formulation excipients and antioxidants indicated in Table 1. Excipients investigated included fillers, disintegrants, buffers, glidants and lubricants. rhIL-11 tablets containing these excipients were prepared by direct compression. Lyophilized rhIL-11 was collected, sieved through #30 mesh screen, and transferred into a suitably sized vial containing all other excipients. Materials were blended by rotating the vial for 2-3 minutes: For those formulations containing magnesium stearate (F1, F2, F4-F8), the magnesium stearate was added at this point and blending was continued for another 0.5-1 minute.

Each tablet weighed 150 mg and contained 2.5 mg of rhIL-11 (added as lyophilized powder prepared by freeze drying the frozen concentrate in vials containing quantities equivalent to 5 mg rhIL-11 as well as sodium phosphate and glycine). The tablets were placed on stability at 40° C./75% RH and tested for strength and % Met⁵⁸ oxidized species at initial, two and four weeks using a reverse phase HPLC method. In general, all the formulations studied showed an increase in % Met⁵⁸ oxidized species. The strength of rhIL-11 in formulation (F3) containing stearic acid dropped from initial 90.4% to 64.1% when placed on stability at 40° C./75% RH for a period of four weeks. In this formulation, % Met⁵⁸ oxidized species also increased from 4.4% to 18.8% during this period. All formulations containing crospovidone (F4, F7, and F8) gave higher initial Met⁵⁸ oxidized species contents as compared to the ones without it (F1). Another 10% increase in Met⁵⁸ oxidized species content was observed in the formulations containing crospovidone after storage for a period of four weeks at 40° C./75% RH.

A second study was designed to examine the potential benefits of antioxidants. The antioxidants evaluated in this study were methionine, ascorbic acid and EDTA. The tablet formulations investigated contained 2.5 mg of rhIL-11 added as concentrate, sodium phosphate, microcrystalline cellulose, and magnesium stearate. Other ingredients are listed in Table 2. Tablets were manufactured by high shear granulation method followed by compression. Tablets were placed on stability at 40° C./75% RH and tested for % Met⁵⁸ oxidized species at initial, two and four week time points. Formulation (W1) containing crospovidone but without any antioxidant produced the highest % Met⁵⁸ oxidized species. Formulations W2, W4, and W5 contained methionine as the antioxidant. These formulations exhibited a small increase of 3-4% in % Met⁵⁸ oxidized species after storage at 40° C./75% RH for period of four weeks. EDTA did not appear to provide additional protection against oxidation (W5). Ascorbic acid was also found not as effective as methionine (W3). Methionine appeared to be the most effective antioxidant in rhIL-11 tablet formulations.

The final tablet formulation was selected based on the results of excipient compatibility and antioxidant studies. Table 3 shows the formula used. In order to prevent the slow drug release of high shear granulation, the rhIL-11 tablets using this formula were manufactured by fluid bed granulation method. The tablets were sealed with a layer of HPMC, enteric coated with an aqueous dispersion containing Eudragit® L30D, talc and triethyl citrate and sealed again with HPMC.

### Example 2

### The Integrity of rhIL-11 Capsules During Tablet Manufacturing

The integrity of rhIL-11 following stresses encountered during the process of tablet manufacturing was investigated. Different compaction forces were used to evaluate the effect of tablet manufacturing stresses on the integrity of rhIL-11. These tablets weighed 150 mg, contained 2.5 mg of rhIL-11 (lyophilized powder), EXPLOTAB®, microcrystalline cellulose, NU-TAB®, syloid and magnesium stearate. Tablets were directly compressed to hardness of 2.4, 4.0, 7.5, or 12.5 KP. The protein integrity was measured by determining % recovery, % multimer, % Met⁵⁸ oxidized species, % related and specific activity of rhIL-11 by T-10 bioassay. The results in Table 4 show that recovery, % multimer, % Met⁵⁸ oxidized species, and % related did not change for rhIL-11 tablets compressed to varying degrees of hardness. Similarly, the specific activity of various formulation blend and tablets were found within the range of specification (Table 5). This shows that compression force does not cause chemical or physical instability of rhIL-11 in the formulations studied.

### Example 3

### Stability of Enteric Coated rhIL-11 Tablets

The stability of enteric coated tablets prepared by fluid bed granulation was tested in HDPE bottles at 40° C./75% RH and room temperature. The stability testing measured % recovery, % Met⁵⁸ oxidized species, and % related species. The results are shown in Table 6. The strength of rhIL-11, % Met⁵⁸ oxidized species and % related species of enteric coated tablet did not change at various time points when stored at room temperature and at 40° C./75% RH.

The dissolution test was performed in a micro-dissolution apparatus using 50 ml of glycine/phosphate dissolution medium at Paddle speed of 50 or 100 rpm. The coated tablets were tested for release of rhIL-11 in 0.1N HCl for two hours followed by glycine/phosphate dissolution medium for the next 60 minutes. The dissolution results revealed that less than 1% rhIL-11 was released in two hours in 0.1N HCl. This suggests that 5% enteric coating is adequate in providing protection against gastric digestion. When dissolution test was run at pH 7 in glycine/phosphate buffer dissolution medium, enteric coating was dissolved and rhIL-11 was released. As seen previously for uncoated tablets, the drug release at 50 rpm was incomplete.

### Example 4

### Direct Compression Formulations

This investigation focused on developing a sustained release tablet formulation that releases IL-11 in about 5 hours. Direct compression formulations were prepared as follows. Lyophilized rhIL-11 was collected, sieved through #30 mesh screen, and transferred into a suitably sized vial containing all other excipients except magnesium stearate. Materials were blended by rotating the vial for 2-3 minutes. Magnesium stearate was added at this point and blending was continued for another 0.5-1 minute. Quantities of final blends equivalent to 2.5 mg rhIL-11 were weighed and compressed using a Kikusowi tableting press. Hardness was adjusted between 7-10 kp.

Dissolution was conducted using the USP paddle method at 50 RPM in 150 ml of phosphate buffer pH 7.0 containing methionine, glycine, and Polysorbate 80 at 37° C. 1 ml samples were withdrawn at predetermined time intervals and replaced with fresh medium. Analysis was conducted at ambient temperatures using a Vydac C4 column (2.1×150 mm, narrow bore). Flow rate was 0.5 ml per minute. Detection was performed at 214 nm. A gradient system was used with 0.1% v/v TFA as mobile phase A and 0.1% v/v TFA in 80% acetonitrile as mobile phase B.

Table 7 shows formulations of tablets prepared by direct compression. Visual evaluation of dissolution of these formulations was performed to characterize their physical behavior in the dissolution medium. Tablets of formulation 1 showed faster erosion than formulations 2 and 3 tablets. Tablets of formulation 2 exhibited the slowest erosion. All formulations exhibited significant swelling. Tablets of formulation 1 exhibited almost complete erosion after 5-6 hours of dissolution. About two thirds of formulation 2 tablets and one third of formulation 3 tablets were eroded over the same period.

One explanation for these results is based on the tablet HPMC content. When HPMC hydrates it forms a gel, which acts as a barrier controlling the dissolution and erosion of the matrix. As HPMC content increases, the gel structure becomes stronger and tighter. This enhances the viscosity and thickness of the gel layer at the surface of the tablet. Consequently, dissolution of the matrix tablet slows down. These results indicate that drug release from formulations 1 and 2 are optimal.

### Example 5

### Wet Granulation Formulations

Wet granulation formulations were prepared using high sheer or fluid bed methods. rhIL-11 solution was added to the excipients except the sustained release polymer and magnesium stearate. The granules were dried, sieved through a #30 mesh screen and blended with the polymer and magnesium stearate. Quantities of final blends equivalent to 2.5 mg rhIL-11 were weighed and compressed using a Kikusowi tableting press. Hardness was adjusted between 7-10 kp.

Dissolution was conducted using USP paddle method at 50 RPM in 150 ml of phosphate buffer pH 7.0 containing methionine, glycine, and Polysorbate 80 at 37° C. 1 ml samples were withdrawn at predetermined time intervals and replaced with fresh medium. Analysis was conducted at ambient temperatures using a Vydac C4 column (2.1×150 mm, narrow bore). Flow rate was 0.5 ml per minute. Detection was performed at 214 nm. A gradient system was used with 0.1% v/v TFA as mobile phase A and 0.1% v/v TFA in 80% acetonitrile as mobile phase B.

Sustained release formulations were prepared using granulation obtained by high sheer technique, see Table 8. A portion of drug solution was added to a blend of all excipients except polymer and magnesium stearate. The wet mass was then dried. The cycle was repeated three times to obtain targeted drug loading. The polymer was then added to the blend followed by the addition of magnesium stearate. The physical behavior of the tablets prepared from these formulations in the dissolution medium was found to be similar to that shown by direct compression formulations containing similar levels of HPMC. Studies with immediate release tablets prepared from high sheer granulation showed that it was difficult to obtain complete release of rhIL-11. Studies with tablets prepared from fluid bed granulation indicate that this method is the most appropriate for rhIL-11 granulation among the techniques that were investigated with respect to manufacture and release of rhIL-11.

Table 9 shows the compositions of three sustained release tablets prepared by fluid bed granulation. Fluid bed granulation contain rhIL-11 mixture, Avicel PH102, sodium phosphate monobasic, sodium phosphate dibasic, methionine and polysorbate 80. In these studies, the sucrose which was used in the direct compression and high sheer granulation formulations was replaced with mannitol, as sucrose was found responsible for discoloring of the immediate release tablets during storage.

### Example 6

### Effect of Buffer Strength on Dissolution of rhIL-11

The effect of buffer strength 50 mM and 100 mM on the dissolution of rhIL-11 was studied. The concentrations of glycine, methionine, and polysorbate 80 in dissolution medium were kept constant. Dissolution of tablets of formulations 6-8 (Table 9), was performed in both media. Dissolution was significantly faster and almost complete in 100 mM medium. On the other hand, only 15% of rhIL-11 were released from the tablet after 5 hours in 50 mM medium.

To understand these results, changes occurring to dissolving tablets were followed in both media. Tablets showed significant swelling and fast erosion in 100 mM medium. They disappeared after about 5 hours of dissolution. On the other hand, tablets swelled in the 50 mM medium but showed minimal erosion after 5 hours of dissolution. This could be due to the fact that the strength of HPMC gel structure is sensitive to ionic strength. Increasing the concentration of phosphate buffer in dissolution medium increases its ionic strength and reduces the strength and tightness of HPMC gel structure.

### Example 7

### Effect of Polymer and its Viscosity Grade

Formulation 6 showed a fast initial dissociation rate in 100 mM phosphate medium. Formulation 6 contains Methocel K4M PREM as a sustained release polymer. In order to reduce this initial rate of dissolution, higher viscosity grade of HPMC (Methocel K15 M PREM) was incorporated in the formulation. Tablets of formulations 7 and 8 exhibited improved dissolution behavior. The higher rate of dissolution exhibited by formulation 8 as compared to that for formulation 7 could be due to the disintegrant properties of the extragranular microcrystalline cellulose (Avicel PH102), which was not present in the tablets of formulation 7.

Matrix tablet formulations were prepared using PEO alone or in combination with HPMC. Visual evaluation of the erosion and dissolution of some of these formulations was encouraging. HPLC analysis of the dissolution samples of these formulations was difficult because of the large molecular weight of PEO.

Prototype formulations which exhibit an optimized release profile for rhIL-11 in 50 mM phosphate medium were prepared and tested. Various formulations were prepared and tested. Monitoring the erosion and dissolution of these formulations indicated that using 20-30% methocel K100 LV, LH, CR Premium as a sustained release polymer might lead to obtaining formulations that exhibit an acceptable dissolution behavior. Table 10 shows the compositions of these formulations.

Dissolution of rhIL-11 from formulations 9 and 10 was examined. The dissolution of rhIL-11 slows down significantly after two hours. Sometimes a decrease in drug concentration was noticed after two hours of dissolution. The incomplete release could be due to adsorption of rhIL-11 to some of the formulation excipients. This phenomenon has also been observed for immediate release tablets and beads.

To improve the release of rhIL-11, buffer species in the formulation as well as the dissolution medium was changed from sodium phosphate to ammonium phosphate. Formulation 11 was prepared using ammonium phosphate while the extragranular sodium phosphates were eliminated from formulation 12. Dissolution of formulations 11 and 12 was conducted in a medium prepared using ammonium phosphate species. Dissolution results showed an increase in the amount of drug released after 5 hours of dissolution while maintaining an acceptable dissolution profile.

### Example 8

### Process for Manufacturing rhIL-11 Delayed Release Multiparticulate Pellets

rhIL-11 enteric-coated pellets are manufactured using a process that includes thawing and dilution of the rhIL-11 drug substance; rhIL-11 layering of the pellets; seal coating; enteric coating; final seal coating; and talc application. The multiparticulate pellet components are listed in Table 11.

rhIL-11 is mixed at room temperature with dilution buffer (4 mM sodium phosphate monobasic, 6 mM sodium phosphate dibasic, 0.3 M glycine, pH 7.0) to a final concentration of 10 mg/ml. The diluted rhIL-11 is compounded with hydroxypropyl methylcellulose (10% solution), methionine, Polysorbate 80, and purified water to generate the drug-layering solution.

The drug-layering solution (˜40,600 g) is applied to ˜20,000 g of sugar spheres within a fluid-bed coater utilizing an inlet temperature range of 47-53° C., an exhaust air temperature of 30-45° C., a supply air velocity of 350-550 CFM, a spray rate of 35-85 g/min, and atomizing air at 30-40 PSI.

A seal-coating solution (˜2900 g) is applied to the drug-layered pellets. The seal-coat solution is composed of a 7.5% solution of hydroxypropyl methylcellulose in purified water (w/w). As with the drug-coating layer, a fluid-bed coater is used utilizing an inlet temperature range of 47-53° C., an exhaust air temperature of 30-55° C., a supply air volume of 400-500 CFM, a spray rate of 25-45 g/min., and atomizing air at 30-40 PSI. The function of this seal coating is to provide an inert barrier between the rhIL-11 protein core and the acidic enteric-coating environment.

An enteric-coating solution (˜30,900 g) is then applied to the sealed drug-coated pellets. A fluid-bed coater is used utilizing an inlet temperature range of 32-38° C., an exhaust air temperature of 25-40° C., a supply air volume of 550-700 CFM, a spray rate of 45-85 g/min., and atomizing air at 25-35 PSI. The function of the enteric-coat layer is to provide a barrier to the acidic pH of the stomach.

A second seal coat (˜3880 g) is applied to the enteric-coated pellets. The seal-coat solution is composed of a 7.5% solution of hydroxypropyl methylcellulose in purified water (w/w). As before, a fluid-bed coater is used utilizing an inlet temperature range of 32-38° C., an exhaust air temperature of 25-40° C., a supply air volume of 550-700 CFM, a spray rate of 25-45 g/min., and atomizing air at 25-35 PSI. The function of the final seal-coat layer is to eliminate potential pellet-to-pellet sticking of the enteric-coat layer. The seal-coat layer is soluble in acid and is removed by the first step in the dissolution test. An in-process strength test is performed after the application of the final seal-coat layer to determine the target fill weight of the capsules.

At the completion of the final seal-coat step, talc is added to the fluid-bed coater. The sealed rhIL-11 enteric-coated pellets are mixed with the talc for 30-60 seconds to eliminate static. The talc-treated pellets are then discharged from the fluid-bed coater and placed into double polyethylene-lined containers with two desiccant bags, one between the polyethylene bags and one outside the bags. The pellets are then filled into capsules.

### Example 9

### Stability of Enteric Coated Multiparticulate Pellets of rhIL-11

The stability of enteric coated multiparticulate pellets (prepared by fluid bed granulation) was tested under long-term storage conditions at 2-8° C. for 0-6 months. The stability testing consisted of strength, % recovery, % Met⁵⁸ oxidized species, and % related species. Table X indicates that strength of rhIL-11, % Met⁵⁸ oxidized species and % related of enteric coated tablet did not change at various time points when stored at 2-8° C. for 0-6 months.

The stability of enteric coated multiparticulate pellets (prepared by fluid bed coating) was tested under accelerated storage conditions at 25° C./60% RH for 0-6 months. These data are presented in Table 13.

### Example 10

### Effect of rhIL-11 Treatment on Chronic Diarrhea in HLA-B27 Rats

Male transgenic HLA-B27 rats were purchased from Taconic Farms (Germantown, N.Y.) and were housed individually under controlled conditions (21° C.; 50±10% humidity; 12-h light/dark cycle). The HLA-B27 rats were obtained at 10 weeks of age and were housed in the animal facility until the age of 40 weeks (350±40 g, n=12). At the age of 40 weeks, the transgenic rats had intestinal inflammation manifested by chronic diarrhea. Age-matched nontransgenic Fisher 344 rats purchased from Charles River Laboratories Inc. (Wilmington, Mass.) genetically engineered to carry high-copy numbers of the human major histocompatibility complex class 1 allele B27 and β₂-microglobulin genes were used as controls (370±20 g, n=6). The Fisher 344 rats appeared to be healthy, and the stool consistency was normal. Loose stools without pellet formation and diarrhea were observed in all HLA-B27 rats prior to administration of rhIL-11.

rhIL-11 multiparticulates contained approximately 1 mg of rhIL-11 per 100 mg multiparticulates, whereas sucrose multiparticulates serves as placebo controls. The cumulative effect of single oral doses of enteric-coated rhILL-11 multiparticulates equivalent to 500 μg/kg rhIL-11 given on alternative days during 2 weeks of treatment was followed by observing the symptoms of diarrhea. Three groups of animals were involved in the study: a test group that included HLA-B27 rats (n=6) treated with rhIL-11; the vehicle-control group consisting of HLA-B27 rats (n=6) treated with placebo; and a healthy control group consisting of age-matched F344 rats (n=6) treated with placebo. The animals were weighed daily during the 2 weeks of oral administration of rhIL-11, and there was no significant change in body weight induced by either rhIL-11 or the placebo.

All HLA-B27 rats showed clinical symptoms of colitis. The stool character was observed daily and characterized as normal, soft, or diarrhea. Scores of 0 for normal, 1 for soft with pellets formed, 2 for soft with no pellet formation, and 3 for diarrhea, were given daily before and during treatment of HLA-B27 rats with rhIL-11 or placebo. Average daily scores were calculated to characterize stool consistency.

Oral administration of rhIL-11 resulted in significant inhibition of the symptoms of diarrhea, i.e., following the first 9 days of treatment the stool character changed toward normal with soft but normally formed pellets. No changes in stool character were observed in HLA-B27 rats receiving placebo. Likewise, placebo treatment had no effect on the normal stool character in healthy F344 rats.

### Example 11

### Effect of rhIL-11 Treatment of HLA-B27 Rats on Intestinal Inflammation

rhIL-11 was administered orally to test animals as described above in Example 10.

Animals were evaluated for intestinal inflammation. All animals were euthanized 4 h after the last administration of rhIL-11 or placebo, and the jejunum and colon were isolated immediately.

Myeloperoxidase (MPO), specifically expressed by neutrophils, is considered a marker of inflammatory cell infiltration. The activity of MPO in intestinal tissue extracts was used as an index of inflammation. Full-thickness jejunal and colonic samples (100-150 mg) were taken from the tissue isolated for the contractile experiments and were immediately frozen in liquid nitrogen. The samples were stored at −80° C. and MPO activity was assayed simultaneously for the whole set of experiments. Homogenization and extraction of MPO from the homogenate were carried out in hexadodecyl-trimethylammonium bromide phosphate buffer (pH 6). MPO activity was tested in 10-μl samples using 3,3′,5,5′-tetramethylbenzidine Microwell peroxidase substrate system (Sigma Chemical Co., St. Louis, Mo.) and horseradish peroxidase as a relative standard. MPO activity was expressed as equivalent to the activity of the relative standard (nanograms of horseradish peroxidase) converting the same amount of 3,3′,5,5′-tetramethylbenzidine substrate for 10 min at room temperature. The data was expressed in nanograms and normalized per gram wet weight of the tissue.

A 2.3-fold increase of MPO activity in the small intestine and a 3.8-fold increase of MPO activity in the colon of HLA-B27 rats treated with placebo in comparison with placebo-treated nontransgenic Fisher 344 rats. Treatment of HLA-B27 rats with rhIL-11 significantly reduced the activity of MPO in both the jejunum and colon. At the end of the 2-week treatment with rhIL-11, MPO activity was reduced to levels that resembled those in nontransgenic Fisher 344 rats. In contest, the same course of treatment with placebo showed no significant decrease in MPO activity in the jejunum and colon from HLA-B27 rats.

### Example 12

### Effect of rhIL-11 Treatment of HLA-B27 Rats on Intestinal Inflammation

### Histological Evaluation

Jejunal and colonic tissue samples were harvested from HLA-B27 rats following the oral administration of rhIL-11 or placebo. The specimens were immersed in 10% neutral-buffered formalin, processed, embedded in paraffin, and sectioned at 5-μm thickness. Slide-mounted sections were stained with hematoxylin and eosin and investigated by light microscopy for the presence of ulceration, inflammatory infiltrates, transmural lesions, and fibrosis. The slides were examined in a blinded fashion, and each parameter was scored as follows: 0 to 2 for ulceration and fibrosis; 0 to 3 for inflammation and depth of lesions. The absence of pathology was scored as zero. A total score was calculated according to the method described by Boughton-Smith et al. (1998) as the sum of the scores of individual parameters (maximum was 10).

The improvement in stool character (seen in Example 10 above) was associated with healing of colonic mucosa. Alternate day therapy with enteric coated rhIL-11 resulted in reduction of the histological lesions in the HLA-B27 transgenic rats. A well established decrease in the histological lesion scores was seen in sections isolated from the colon of animals receiving rhIL-11.

### Example 13

### Acute Effect of rhIL-11 on Basal Contractile Activity

Segments of the jejunum (approximately 5 cm distal to the ligament of Treitz) and the colon (approximately 4 cm distal to the ileocecal junction) were harvested and placed in ice-cold oxygenated Krebs' bicarbonate solution. Longitudinal muscle strips were dissected from the intestinal segments by gently peeling the muscle in longitudinal direction. Muscle strips (10-12 mm long) were excised following the direction of the muscle with the help of a dissecting microscope, and both ends were secured with silk surgical suture (size 3-0). The strips were mounted vertically in 10-ml organ baths with one end fixed and the other attached to an isometric force transducer (Radnoti Glass Technology Inc., Monrovia, Calif.). The baths were filled with Krebs' bicarbonate solution, maintained at 37° C. and aerated with 95% O₂ and 5% CO₂. The solution was changed by perfusion at 30-min intervals. Each smooth muscle strip was allowed to equilibrate at zero tension for 20 min, followed by consecutive loading with 0.20 g force increments until a level of optimal resting tension was achieved. Resting tension was considered to increase with loading. Strips were allowed an additional 20 min of equilibration. All experiments were performed at optimal tension and isometric contractions were recorded using a MacLab data acquisition system (AD Instruments Ltd., Castle Hill, Australia).

In the jejunal longitudinal muscle of F344 control rats, basal activity recorded at optimal tension was characterized by low resting tension (3.1±0.8 nM/mm²) and spontaneous low-amplitude contractions appearing at a frequency of 18±5 cycles/min. There was no significant difference between the basal activity recorded in muscled isolated from placebo-treated F344, placebo-treated HLA-B27 rats, or HLA-B27 rats treated with rhIL-11. When rhIL-11 (1-10,000 ng/ml) was added to the bathing solution, no significant changes in background activity were found in jejunal muscles isolated from both Fisher 344 or HLA-B27 rats. Accordingly, contractions induced by carbachol (0.1 μM) were not altered when rhIL-11 (1-10,000 ng/ml) was present into the bathing solution.

Colonic longitudinal muscles isolated from placebo-treated control F344 rats showed low resting tension (2.4±0.3 mN/mm²) with or without occurrence of spontaneous contractions. Resting tension and spontaneous contractions were similar in muscles from F344 and HLA-B27 rats receiving placebo or rhIL-11. The addition of rhIL-11 (1-10,000 ng/ml) to the bathing solution showed no acute effects on spontaneous contractility or contractile responses to carbachol (1 μM) in the colon of Fisher 344 rats or HLA-B27 rats.

### Example 14

### Effects of rhIL-11 Treatment on Receptor-Independent Intestinal Muscle Contraction

The effect of rhIL-11 treatment on receptor-independent intestinal muscle contraction was examined. Increasing the concentration of KCl in the bathing solution induced receptor-independent membrane depolarization and muscle contraction. Concentrations of 60 to 80 mM KCl were required to elicit maximal contractions in jejunal or colonic muscle strips isolated from both Fisher 344 and HLA-B27 rats. However, the active tension generated by muscles from placebo-treated HLA-B27 rats was lower compared with that generated by muscles from placebo-treated Fisher 344 rats. Treatment of HLA-B27 rats with rhIL-11 increased the maximal contraction induced by high KCl in both the jejunum and colon. Moreover, there was no significant difference between the responses to high KCl in muscles isolated from HLA-B27 rats treated with rhIL-11 compared with placebo-treated Fisher 344 rats.

### Example 15

### Effects of rhIL-11 Treatment on Cholinergic Intestinal Muscle Contraction

The effect of rhIL-11 treatment on cholinergic intestinal muscle contraction was examined. Complete dose-response curved to carbachol were obtained in jejunal and colonic longitudinal muscle. Longitudinal muscles isolated from the jejunum of HLA-B27 rats showed abnormal contractile responses. The maximal active tension generated in response to increasing concentrations of carbachol (a nM-10 μM) was significantly lower in the muscles isolated from placebo-treated HLA-B27 rats compared with placebo-treated Fisher 344 rats. The reduction in contractile responses was accompanied by a shift of the dose-response curve to lower carbachol concentrations. Accordingly, the EC₅₀ for carbachol in jejunal muscles from placebo-treated HLA-B27 rats is significantly lower compared with the EC₅₀ value obtained in the jejunum of Fisher 344 rats. The treatment of HLA-B27 transgenic rats with rhIL-11 resulted in a significant increase in carbachol-induced maximal tension generated by the jejunal muscle. Besides the significant increase, the amplitude of maximal response remained lower than the maximal contraction in muscles from placebo-treated Fisher 344 rats. The EC₅₀ for carbachol in the jejunum of HLA-B27 rats treated with rhIL-11 was significantly reduced compared with placebo-treated HLA-B27 rats and was similar to the EC₅₀ in the jejunum of Fisher 344 rats.

The maximal active tension generated in response to carbachol by colonic muscles from placebo-treated HLA-B27 rats was lower than that generated by muscles from placebo-treated Fisher 344 rats. Following rhIL-11 therapy, the maximal tension induced by carbachol in colonic muscles from rhIL-11 treated HLA-B27 rats was significantly increased compared with placebo-treated HLA-B27 rats and was similar to that in the colon of placebo-treated Fisher 344 rats. In contrast to the jejunum, the concentration-effect curves for carbachol obtained in colonic muscles from F344 and HLA-B27 rats treated with placebo, as well as from HLA-B27 rats treated with rhIL-11, had similar position and did not show significant difference between EC₅₀ values.

### Example 16

### Effects of rhIL-11 Treatment on Neurally Mediated Intestinal Muscle Contraction

In the longitudinal muscle of the jejunum, EFS (0.5-ms pulse duration, 5 Hz, 5-s train duration) induced contractile responses. The increase in tension reached maximum during stimulation and decreased to the resting level after the end of the stimulus train. Responses to EFS were reproducible throughout the experiment. In the presence of atropine (1 μM) and guanethidine (10 μM), EFS induced nonadrenergic, noncholinergic (NANC) contractile responses of lower amplitude. No relaxation was observed. Guanethidine alone had no effect on EFS-induced contractions; thus, the difference between the control response and the NANC component represented a cholinergic (atropine-sensitive) component of the EFS-induced contraction. The effects of rhIL-11 therapy on control and NANC neurally mediated contractions were examined. Control responses to EFS obtained in jejunal muscles from placebo-treated HLA-B27 rats had lower amplitude compared with placebo-treated Fisher 344 rats, whereas there was no significant difference between the amplitude of NANC contractions. Oral treatment of HLA-B27 rats with rhIL-11 normalized the amplitude of control EFS-induced contraction and had no significant effect on the NANC response. Tetrodotoxin (1 μM) completely abolished both control and NANC responses to EFS, indicating that they result from activation of enteric neurons.

In colonic muscles, EFS induced a contractile response, which was partially inhibited by atropine and guanethidine, revealing a NANC contraction. Similar to the jejunum, the colonic muscles maintained a relatively low level of resting tension, and no relaxatory responses were observed. In muscles from placebo-treated HLA-B27 rats, the control response to EFS was reduced compared with placebo-treated F344 rats. In contrast to the jejunum, there was also a significant reduction in the amplitude of NANC contractions. Treatment of HLA-B27 rats with rhIL-11 significantly increased the amplitude of control EFS-induced contraction and normalized the NANC response. Despite the recovery, the treated HLA-B27 rats remained lower compared with placebo-treated F344 rats. Both control and NANC contractions induced by EFS were abolished by tetrodotoxin (1 μM).

### Other Embodiments

Other embodiments are within the claims.

TABLE 1
Formulations Used in Excipient Compatibility Study

| No | Test Excipient (%) | Other Ingredients (%) |
|---|---|---|
| F1 | Control | Avicel PH 112 (77), Explotab (8), Syloid |
| | | (0.25), Mg Stearate (0.25) |
| F2 | Talc (0.25) | Avicel PH 112 (77), Explotab (8), Mg |
| | | Stearate (0.25) |
| F3 | Stearic Acid (1) | Avicel PH 112 (76), Explotab (8), Syloid |
| | | (0.25) |
| F4 | Crospovidone (5) | Avicel PH 112 (80), Syloid (0.25), Mg |
| | | Stearate (0.25) |
| F5 | Nu-Tab (77) | Explotab (8), Syloid (0.25), Mg. Stearate |
| | | (0.25) |
| F6 | Avicel PH 112 (38.6), | Explotab (8), Syloid (0.25), Mg. Stearate |
| | Nu-Tab (38.6) | (0.25) |
| F7 | Nu-Tab (39.9), | Avicel PH 112 (39.9), Syloid (0.25), Mg. |
| | Crospovidone (5) | Stearate (0.25) |
| F8 | Nu-Tab (40), | Avicel PH 112 (40), Mg. Stearate (0.5) |
| | Crospovidone (5), no | |
| | Syloid | |

TABLE 2
Formulations Investigated to Select Anti-Oxidant

| | | | Tablet |
|---|---|---|---|
| No. | Antioxidant (%) | Other Excipient | Wt (mg) |
| W1 | None | Crospovidone | 200 |
| W2 | Methionine (0.5) | Crospovidone | 20 |
| W3 | Ascorbic Acid (1) | Crospovidone | 200 |
| W4 | Methionine (0.5) | None | 250 |
| W5 | Methionine (0.5) | None | 250 |
| | (EDTA) (0.8) | | |

TABLE 3
rhIL-11 Leading Tablet Formulation
Manufactured by Fluid Bed Granulation

| Ingredients | mg/tablet |
|---|---|
| Intragranular | |
| rhIL-11 | 5.561 |
| (concentrate equivalent to 2.5 mg) | |
| Avicel PH 102 | 92.50 |
| Na₂HPO₄ Anhydrous | 8.50 |
| NaH₂PO₄ Anhydrous | 6.50 |
| Methionine | 1.00 |
| Tween 80 | 0.339 |
| Extragranular | |
| Avicel PH 112 | 73.5 |
| Na₂HPO₄ Anhydrous | 4.25 |
| NaH₂PO₄ Anhydrous | 3.25 |
| Explotab | 4.00 |
| Magnesium Stearate | 0.60 |
| Total | 200 |
| Coating | |
| Eudragit L30D | 5% |

TABLE 4
Effect of Physical Stress on the Integrity of rhIL-11

| Hardness | Recovery^{a} | Multimer^{b} | Met⁵⁸ | Related |
|---|---|---|---|---|
| (Kp) | (%) | (%) | (%) | (%) |
| 2.4 | 111.0 | 0.2 | 4.1 | 3.7 |
| 4.0 | 105.3 | 0.3 | 4.2 | 3.9 |
| 7.5 | 96.4 | 0.3 | 4.4 | 4.1 |
| 12.8 | 100.2 | 0.2 | 4.3 | 4.0 |

^{a}Measured by RP-HPLC
^{b}measured by Size Exclusion Chromatography.

TABLE 5
In Vitro Bio-activity by T-10 bioassay
(Directly compressed tablets of rhIL-11)

| | Sp Act | IC Sp Act |
|---|---|---|
| Formulation | Uwho/mg | Uwho/mg |
| Tablet: Crospovidone, Syloid, | 5.82E+06 | 6.70E+06 |
| Avicel, Mg Stearate | | |
| Blend: Avicel, Nu-Tab, Explotab, | 6.57E+06 | 5.80E+06 |
| Syloid, Mg Stearate | | |
| Tablet: Avicel, Nu-Tab, Explotab, | 6.38E+06 | 7.70E+06 |
| Syloid, Mg Stearate | | |

Sp Act: Specific Activity;
IC Sp Act: Internal Control Specific Activity

TABLE 6
Stability of Enteric Coated Tablets of rhIL-11
(by Fluid Bed Granulation)

| | | | Related |
|---|---|---|---|
| Time (Weeks) | Strength | Met⁵⁸ | Species |
| (Conditions) | (%) | (%) | (%) |
| Initial | 93.6 | 5.0 | 6.7 |
| 2 | 86.9 | 4.5 | 3.4 |
| (40° C./75% RH) | | | |
| 4 | 86.6 | 5.0 | 3.8 |
| (40° C./75% RH) | | | |
| 15 | 94.1 | 4.0 | 4.9 |
| (Room Temp.) | | | |

TABLE 7
Sustained Release Tablet Formulations
Prepared by Direct Compression

| | Formulation 1 | Formulation | Formulation |
|---|---|---|---|
| Ingredients | (%) | 2 (%) | 3 (%) |
| Lyophilized rhIL-11* | 6.3 | 6.0 | 5.7 |
| HPMC (Methocel K4M | 10.5 | 15 | 19 |
| PREM) | | | |
| Microcrystalline Cellulose | 10.5 | 10 | 9.5 |
| (Avicel PH112) | | | |
| Sucrose (NU-TAB ®) | 68.5 | 65 | 62 |
| Silicon Dioxide (Syloid) | 0.26 | 0.25 | 0.24 |
| Mg-stearate | 0.79 | 0.75 | 0.71 |
| Na₂HPO₄ (Anhydrous) | 1.78 | 1.7 | 1.62 |
| NaH₂PO₄ (Anhydrous) | 1.37 | 1.3 | 1.24 |

*Each tablet contains 2.5 mg rhIL-11.

TABLE 8
Composition of Sustained Release Tablet
Formulations Prepared by High Sheer Wet Granulation

| | Formulation 4 | Formulation 5 |
|---|---|---|
| Ingredients | (%) | (%) |
| rhIL-11* | 1.0 | 1.0 |
| Methocel K4M PREM | 10.0 | 15.0 |
| Avicel PH112 | 30.0 | 30.0 |
| NU-TAB ® | 55.04 | 50.04 |
| Syloid | 0.25 | 0.25 |
| Mg-stearate | 0.74 | 0.74 |
| Na₂HPO₄ (Anhydrous) | 1.68 | 1.68 |
| NaH₂PO₄ (Anhydrous) | 1.29 | 1.29 |

*Each tablet contains 2.5 mg rhIL-11 added as bulk solution.

TABLE 9
Composition of Sustained Release Tablet
Formulations Prepared by Fluid Bed Granulation Using
Higher Viscosity Grades of HPMC

| | Formulation 6 | Formulation 7 | Formulation 8 |
|---|---|---|---|
| Ingredients | (%) | (%) | (%) |
| rhIL-11 Granules* | 48.6 | 45.7 | 45.7 |
| Methocel K4M | 31.9 | 25 | 24 |
| PREM | | | |
| Methocel K15M | — | 5.3 | 6.0 |
| PREM | | | |
| Mannitol | 18.44 | 23.0 | 15.3 |
| Avicel PH102 | — | — | 8.0 |
| Syloid | 0.26 | 0.25 | 0.25 |
| Mg-Stearate | 0.8 | 0.75 | 0.75 |

*Prepared by fluid bed granulation. Equivalent to 2.5 mg rhIL-11 per tablet.

TABLE 10
Composition of Sustained Release Tablet
Formualtions Prepared by Fluid Bed Granulation Using
Lower viscosity grades of HPMC and various phosphate
buffer species

| | Formulation | Formulation | Formulation | Formulation |
|---|---|---|---|---|
| Ingredients | 9 (%) | 10 (%) | 11 (%) | 12 (%) |
| rhIL-11 | 45.7 | 45.7 | 45.7 | 45.7 |
| Granules* | | | | |
| Methocel K100 | 25.0 | 30.0 | 25 | 25 |
| LV, LH, CR, | | | | |
| Premium | | | | |
| Mannitol | 16.3 | — | — | 28.3 |
| Syloid | 0.25 | 0.25 | 0.25 | 0.25 |
| Mg-Stearate | 0.75 | 0.75 | 0.75 | 0.75 |
| Na₂HPO₄ | 6.8 | 13.3 | — | — |
| NaH₂PO₄ | 5.2 | 10 | — | — |
| (NH4)₂HPO₄ | — | — | 16.1 | — |
| (NH4)H₂PO₄ | — | — | 12.2 | — |

*Prepared by fluid bed granulation. Equivalent to 2.5 mg rhIL-11

TABLE 11
Composition of IL-11 Delayed Release Multiparticulate Capsules

| | | Target for | |
|---|---|---|---|
| | Percentage | 5 mg | |
| Component | (% wt/wt) | Capsul (mg) | |
| rhIL-11 | 1.10^{b} | 5.500 | |
| Sugar spheres, NF | 68.0 | 339.9 | |
| Glycine, USP | 2.47 | 12.38 | |
| Sodium phosphate (dibasic), USP | 0.180 | 0.8855 | |
| Sodium phosphate (monobasic), USP | 0.060 | 0.3037 | |
| Polysorbate-80, NF | 0.028 | 0.1377 | |
| Methionine, USP | 0.206 | 1.028 | |
| Hydroxypropyl methylcellulose, USP | 3.91 | 19.57 | |
| Methacrylic acid copolymer dispersion, | 15.0 | 74.95 | |
| NF (Eudragit L30D-55) | | | |
| Talc, USP | 7.50 | 37.49 | |
| Sodium hydroxide, NF | 0.090 | 0.4496 | |
| Triethyl citrate, NF | 1.50 | 7.490 | |
| Purified water, USP | Removed during | q.s. | |
| | processing | | |
| Size #0 Hard gelatin capsule | | | |
| Total | | 500 | mg |

A 10% overage rhIL-11 is used to compensate for losses during
manufacture.

Label/Package

TABLE 12
rhIL-11 Delayed Release Capsules, 5 mg/Capsule
Long Term Storage at 2-8° C., 0-18 Months

| | | | | Impurities & | | Dissolution - Acid | Dissolution | |
|---|---|---|---|---|---|---|---|---|
| | | Total | Met⁵⁸- | rhIL-11 | | Stage | Buffer Stage | |
| | | Inactive | Oxidized | Related | Specific Activity | (0.1 N HCl) | (Phosphate Buffer) | |
| Tests | Strength | Species | Species | Species | (T-10 Bioassay) | Average | Average | Moisture |
| Initial | 4.60 | 9.4% | 6.7% | 2.7% | 8.1 × 10⁶ | 3% | 76% | 1.1% |
| 1 Month | 4.94 | 7.1% | 4.5% | 2.7% | NS^{b} | 3% | 74% | 1.3% |
| 83 Days | 4.94 | 6.3% | 4.0% | 2.3% | 7.0 × 10⁶ | 3% | 82% | 1.1% |
| 6 Months | 4.74 | 7.3% | 4.4% | 3.0% | 7.0 × 10⁶ | 2% | 84% | 1.1% |
| 9 Months | 5.02 | 8.1% | 5.3% | 2.8% | 1.1 × 10⁷ | 3% | 65% | 2.4% |
| 12 Months | 4.49 | 5.0% | 3.2% | 1.9% | 8.9 × 10⁶ | NT | NT | 1.6% |
| 18 Months | 4.60 | 5.8% | 4.0% | 1.8% | 8.0 × 10⁶ | 1% | 69% | 1.1% |

TABLE 13
rhIL-11 Delayed Release Capsules, 5 mg/Capsule
Long Term Storage at 25° C., 0-18 Months

| | | | | Impurities & | | Dissolution - Acid | Dissolution | |
|---|---|---|---|---|---|---|---|---|
| | | Total | Met⁵⁸- | rhIL-11 | | Stage | Buffer Stage | |
| | | Inactive | Oxidized | Related | Specific Activity | (0.1 N HCl) | (Phosphate Buffer) | |
| Tests | Strength | Species | Species | Species | (T-10 Bioassay) | Average | Average | Moisture |
| Initial | 4.60 | 9.4% | 6.7% | 2.7% | 8.1 × 10⁶ | 3% | 76% | 1.1% |
| 1 Month | 4.86 | 7.3% | 4.6% | 2.7% | NS^{b} | 2% | 76% | 1.4% |
| 83 Days | 4.82 | 6.6% | 4.0% | 2.5% | 6.9 × 10⁶ | 3% | 80% | 1.2% |
| 6 Months | 4.75 | 9.1% | 5.4% | 3.7% | 5.7 × 10⁶ | 1% | 75% | 1.2% |
| 9 Months | 4.87 | 10.3% | 6.7% | 3.6% | 1.2 × 10⁷ | 2% | 65% | 1.5% |
| 12 Months | 4.48 | 7.9% | 5.1% | 2.9% | 7.4 × 10⁶ | 2% | 68% | 2.1% |

What is claimed is:

1. A pharmaceutical composition comprising a therapeutically effective delayed release oral dosage form of a bioactive polypeptide, wherein said composition comprises
a bioactive polypeptide, wherein said polypeptide includes one or more properties selected from the group consisting of lacking an N-linked glycosylation site, having no more than one cysteine amino acid, and having a basic pI;
at least one binder;
at least one plasticizer;
at least one glidant; and
a methacrylic acid copolymer.

2. The composition of claim 1, wherein said polypeptide includes two or more properties selected from the group consisting of lacking an N-linked glycosylation site, having no more than one cysteine amino acid, and having a basic pI.

3. The composition of claim 1, wherein said polypeptide lacks an N-linked glycosylation site, having no more than one cysteine amino acid, and having a basic pI.

4. The composition of claim 1, wherein said polypeptide has no cysteine amino acids.

5. A pharmaceutical composition comprising a therapeutically effective delayed release oral dosage form of an interleukin-11 (“IL-11”) polypeptide, wherein said composition comprises
an IL-11 polypeptide;
at least one binder;
at least one plasticizer;
at least one glidant; and
a methacrylic acid copolymer.

6. The pharmaceutical composition of claim 5, further comprising a carbohydrate.

7. The pharmaceutical composition of claim 6, wherein said carbohydrate comprises sucrose.

8. The pharmaceutical composition of claim 6, wherein said carbohydrate is present in said pharmaceutical composition at 60%-75% wt/wt.

9. The pharmaceutical composition of claim 9, further comprising glycine.

10. The pharmaceutical composition of claim 9, wherein said glycine is present in said pharmaceutical composition at 1% to 4% wt/wt.

11. The pharmaceutical composition of claim 9, further comprising methionine.

12. The pharmaceutical composition of claim 11, wherein methionine is present in said composition at a concentration of 0.1% to 0.5% wt/wt.

13. The pharmaceutical composition of claim 1, wherein said methacrylic acid copolymer is a pH dependent anionic polymer solubilizing above pH 5.5.

14. The pharmaceutical composition of claim 13, wherein said methacrylic acid copolymer is provided as a dispersion.

15. The pharmaceutical composition of claim 13, wherein said methacrylic acid copolymer is presenting in said pharmaceutical composition at a concentration of 10% to 20% wt/wt.

16. The pharmaceutical composition of claim 9, wherein said IL-11 polypeptide has the amino acid sequence of a human IL-11 polypeptide.

17. The pharmaceutical composition of claim 9, wherein said IL-11 polypeptide is a recombinantly produced IL-11 polypeptide.

18. The pharmaceutical composition of claim 16, wherein said IL-11 polypeptide is a recombinantly produced IL-11 polypeptide.

19. The pharmaceutical composition of claim 5, wherein said at least one binder is hydroxypropyl methylcellulose (HPMC).

20. The pharmaceutical composition of claim 5, wherein HPMC is present in said composition at a concentration of 3%-7%.

21. The pharmaceutical composition of claim 5, wherein said at least one glidant is talc.

22. The pharmaceutical composition of claim 21, wherein talc is present in said composition at a concentration of 5% to 10%.

23. The pharmaceutical composition of claim 5, wherein said at least one plasticizer is triethyl citrate or polysorbate-80.

24. The pharmaceutical composition of claim 23, wherein said triethyl citrate is present in said composition at a concentration of 1%-2% wt/wt.

25. The pharmaceutical composition of claim 23, wherein said polysorbate-80 is present in said composition at a concentration of 0.015%-0.045% wt/wt.

26. The pharmaceutical composition of claim 5, wherein said at least one plasticizer is triethyl citrate.

27. A pharmaceutical composition comprising a therapeutically effective delayed release oral dosage form of a bioactive polypeptide,
wherein said bioactive polypeptide includes one or more properties selected from the group consisting of lacking an N-linked glycosylation site, having no more than one cysteine amino acid, and having a basic pI, and
wherein said bioactive polypeptide is substantially enveloped by a first sealing coat, an enteric coating layer, and a second sealing coat, wherein said enteric coating layer is substantially disposed between said first and second sealing coat.

28. A pharmaceutical composition comprising a therapeutically effective delayed release oral dosage form of an Interleukin-11 (“IL-11”) polypeptide, wherein said IL-11 polypeptide is substantially enveloped by a first sealing coat, an enteric coating layer, and a second sealing coat, wherein said enteric coating layer is substantially disposed between said first and second sealing coat.

29. The pharmaceutical composition of claim 28, wherein at least one of said first sealing coat and said second sealing coat is HPMC.

30. The pharmaceutical composition of claim 28, wherein said first sealing coat and said second sealing coat comprise HPMC.

31. The pharmaceutical composition of claim 28, wherein said enteric coating layer comprises a methacrylic acid copolymer.

32. The pharmaceutical composition of claim 28, wherein said IL-11 polypeptide is provided disposed on a carbohydrate.

33. The pharmaceutical composition of claim 32, wherein said carbohydrate is sucrose.

34. The pharmaceutical composition of claim 28, further comprising methionine.

35. The pharmaceutical composition of claim 28, further comprising glycine.

36. The pharmaceutical composition of claim 28, further comprising a glidant.

37. The pharmaceutical composition of claim 36, wherein said glidant is talc.

38. The pharmaceutical composition of claim 28, wherein said composition is provided as a capsule or a tablet.

39. The pharmaceutical composition of claim 38, wherein said composition is provided as a tablet.

40. The pharmaceutical composition of claim 38, wherein said composition is provided as a capsule.

41. The pharmaceutical composition of claim 40, wherein said capsule is a gelatin capsule.

42. A method of delivering a bioactive polypeptide to a subject, the method comprising orally administering to said subject the pharmaceutical composition of claim 1 in an amount sufficient to elicit a biological response in said subject.

43. A method of delivering an interleukin-11 (“IL-11”) polypeptide to a subject, the method comprising orally administering to said subject the pharmaceutical composition of claim 5 in an amount sufficient to elicit a biological response in said subject.

44. The method of claim 43, wherein said IL-11 polypeptide elicits a biological response in the small intestine of said subject.

45. The method of claim 43, wherein said subject is a human.

46. The method of claim 43, wherein said IL-11 polypeptide is administered in a composition comprising
at least one binder;
at least one plasticizer;
at least one glidant; and
a methacrylic acid copolymer.

47. The method of claim 43, wherein said interleukin-11 (IL-11) polypeptide is recombinant human IL-11.

48. A method of treating inflammatory bowel disease in a subject, the method comprising orally administering to a subject in need thereof a therapeutically effective dose of IL-11.

49. The method of claim 48, wherein said inflammatory disease is ulcerative colitis.

50. The method of claim 48, wherein said inflammatory disease is Crohn's disease.

51. The method of claim 48, wherein said subject is a human.

52. The method of claim 48, wherein said IL-11 polypeptide is administered in a composition comprising
at least one binder;
at least one plasticizer;
at least one glidant; and
a methacrylic acid copolymer.
